(19) United States
(12) Patent Application Publication
(10) Pub. No.: US 2006/0029675 A1
(43) Pub. Date: Feb. 9, 2006

(54) Method for coating substrate with antimicrobial agent and product formed thereby

(75) Inventor: Devin C. Ginther, Converse, TX (US)

Correspondence Address: Kinetic Concepts, Inc.;Attn: Legal Dept., P.O. Box 659508, San Antonio, TX, 78265-9508, US

(73) Assignee: KCI Licensing, Inc., San Antonio, TX (US)

(21) Appl. No.: 11/189,195

(22) Filed: Jul. 26, 2005

Related U.S. Application Data
(60) Provisional application No. 60/591,014, filed on Jul. 26, 2004.

(51) Int. Cl. A61K 9/14; A61K 33/38

(52) U.S. Cl. 424/486; 424/618

(57) ABSTRACT

A method for uniformly coating a foam or dressing with antimicrobial polymer incorporating agents, such as silver, and a foam or dressing formed by this process. Such foam or dressing is particularly useful in combination with negative pressure wound therapy.

### CROSS REFERENCE TO RELATED APPLICATIONS

The present application claims priority to U.S. Provisional Patent Application No. 60/591,014, filed Jul. 26, 2004, the disclosure of which is incorporated herein by reference.

### BACKGROUND OF THE INVENTION

1. Field of the Invention

This invention relates in general to a process for coating reticulated foam, and more particularly but not by way of limitation, to a method for coating reticulated foam with antimicrobial agents that provides uniform coating throughout the reticulated foam and the product formed by the method.

2. Description of the Related Art

A wide variety of novel and/or established antimicrobial compounds combined with wound dressing can control microbial contamination and potentially lower the rate of infection. The coating uniformity is an essential key to the antimicrobial performance of the wound dressing. What is not known is any method of coating medical wound dressings or foams wherein the entire volume of the dressing is capable of uniform coating with a polymer coating system. This occurs for several reasons.

Particularly, certain foams are very thick, often in the range of about 1.25 inches. The thickness of these dressings limits the coating process, inasmuch as there is no way to insure a uniform coating throughout the entire structure such that the structure is capable of being severed omni-directionally while still having the desired anti-microbial agent exposed for use in a wound.

Certain coating methods exist, such as vapor deposition (both physical and chemical), electrostatic coating, spraying and sputter coating. However, these coating methods are costly, and are not adaptable to uniformly coating three-dimensional surfaces of certain dressings, such as reticulated foam. In addition, these methods have extensive environmental issues that concern users of the dressings in the medical industry.

Other methods of adding antimicrobials to the dressing, such as additives in the foaming process itself or the use of adjunctive therapies or combination products (e.g. on thin antimicrobial dressing attached to the foam) exist, but are difficult to use. Particularly these methods are known to mechanically impact the foam and to materially impact the permeability of the foam.

Because wound sizes and shapes have almost infinite variations, the wound dressing must be adaptable to accommodate the wound and provide appropriate anti-microbial properties to prevent further infection. Accordingly, there is a need to develop a process for uniformly coating the dressing or foam with anti-microbial agents sufficient to decontaminate the wound yet simple to use and cost-effective, such that the foam will be adapted for in situ adjustment to match the wound shape and dimension.

### BRIEF SUMMARY OF THE INVENTION

The present invention fulfills this and other needs through the development of a process for uniformly coating a foam or dressing and a foam or dressing formed by this process with an antimicrobial polymer. Such foam or dressing is particularly useful in negative pressure wound therapy.

### BRIEF DESCRIPTION OF THE SEVERAL VIEWS OF THE DRAWING

A more complete understanding of the method and apparatus of the present invention may be obtained by reference to the following Detailed Description of the Invention, with like reference numerals denoting like elements, when taken in conjunction with the accompanying Drawings wherein:

FIG. 1 is a flow chart of a process for uniformly coating a wound dressing with antimicrobial agents;

FIG. 2 is a schematic diagram of certain steps of the process of FIG. 1;

FIG. 3 is a schematic top plan view of a dressing coated using the process of FIG. 1 as applied to a wound site;

FIG. 4 is a side view of the dressing of FIG. 3 on a wound site in combination with a negative pressure therapeutic device; and

FIG. 5 is a cross section of the dressing of FIG. 3 taken along line 5-5, illustrating the uniform coating of the dressing.

### DETAILED DESCRIPTION OF THE INVENTION

The present invention provides a method for uniformly coating a wound dressing with antimicrobial polymers incorporating agents, such as Au, utilizing a novel process and a wound dressing formed under the process. The method of uniform coating enables a user of the dressing to sever the dressing in any direction and still have all exposed surfaces uniformly coated with the antimicrobial agent sufficient to decontaminate the wound.

A polyurethane foam is uniformly coated with a silver hydrogel polymer. The polymer coating itself contains PVP or Poly[vinlypyrrolidine], which is a water-soluble polymer with pyrroloidone side groups, typically used as a food additive, stabilizer, clarifying agent, tableting adjunct and dispersing agent. It is most commonly known as the polymer component of Betadine (a povidone-iodine formulation). In addition, the coating may contain Chitosan, which is a deacetylated derivative of chitin, a polysaccharide that is refined from shells of shrimps, crabs and other crustaceans. Chitosan has also been used in hemostatic dressings. The third optional component of the polymer is preferably Silver Sodium Aluminosilicate, which is silver salt powder with 20% active ionic silver by weight.

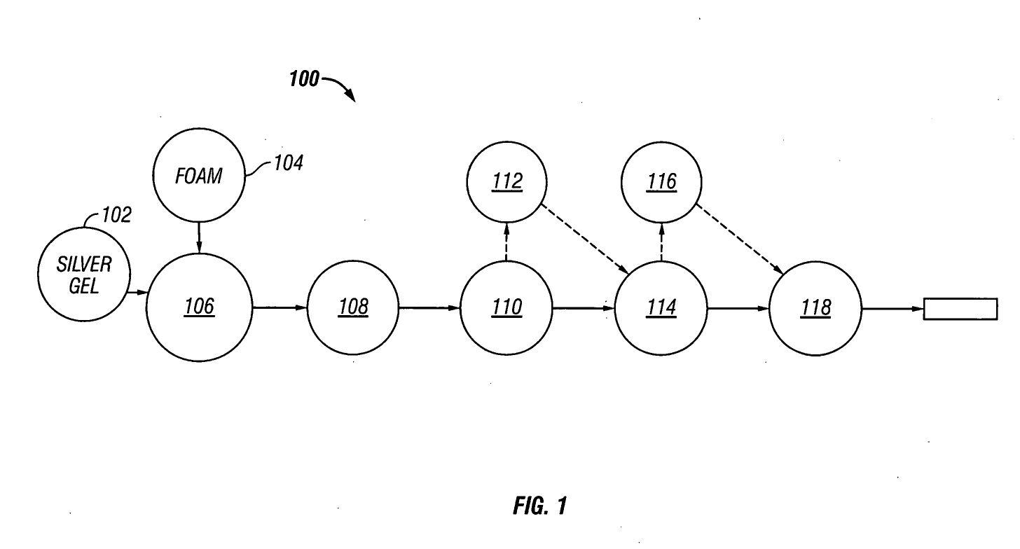

Referring first to FIG. 1, a method 100 for impregnating a foam with a silver polymer coating or antimicrobial coating is shown in the flow chart. First, a hydrophilic gel is combined with silver to create a coating solution, 102. The solution is then placed in a holding tank and continuously agitated in a closed, dark environment, 104. The dark environment is optional, but is included because of the light-sensitivity of silver. In a light-exposed environment, the foam may change color, which results in a non-aesthetic appearance. The foam, which may comprise reticulated polyurethane die cut, is placed in the holding tank, 106. The foam is then saturated with the solution, which is accomplished through soaking or squeezing the foam, 108. Next, excess solution is removed from the foam, 110. Roller nips or similar devices may be utilized to control the amount of solution removed from the foam. Optionally, the weight of the saturated foam, while still wet, may be calculated, 112.

The foam is then placed in a convectional forced-air oven set to a predetermined temperature and time to completely dry the solution-coated foam, 114. Alternatively, to verify the dry condition of the foam, the weight of the foam may be checked again, 116. If light-sensitivity remains an issue, the foam can be packaged in a moisture vapor transmission rate (MVTR) pouch, which limits the exposure of the foam to light and to humidity, 118. The foam is now ready for use on such sites as partial thickness burns, traumatic wounds, surgical wounds, dehisced wounds, diabetic wounds, pressure ulcers, leg ulcers, flaps and grafts.

In one example, a foam made by the method described as achieved in-vitro efficacy on two common bacteria—staphylococcus aureus and pseudomonas aeruginosa, with a 20% silver salt load (4% silver by weight, though about 0.1% to about 6% has shown to be at least partially effective). The dressing maintains its effectiveness for 72 hours through a controlled and steady state release of ionic silver. Specifically, a diffusive gradient exists between the silver coating and the anionic rich outside environment that lead to disassociation and eventual transport of the silver ion. Using the above process, over a 6 log reduction or about 99.9999% of pathogenic bacteria have been eliminated between about 24 hours and about 72 hours.

The coating process can easily incorporate other additives, such as enzymatic debriders, anesthesia agents, growth factors and many other biopharmaceuticals. In addition, the coating can be formulated specific to coat thickness, although very thin coatings (about 2 to 10 micrometers) are preferable. The formulation can further be adapted to allow for large particle sizes and different release kinetics, such as concentration and rate and the duration of release.

The uniform and impregnated coating allows for delivery of silver ions both outside and within the foam. In this manner, not only is bacteria eliminated on the wound bed, but also within the dressing itself. This is particularly useful when using the dressing in combination with a negative pressure therapy. Also, odor reduction is an added benefit of this method.

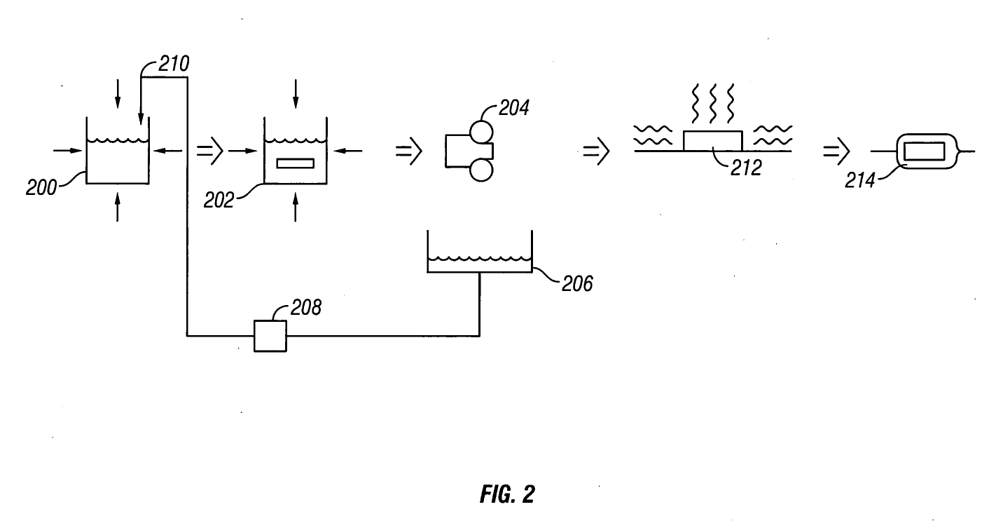

Referring now to FIG. 2, a schematic diagram of certain steps of the process 100 of FIG. 1 is shown. First, the solution of hydrophilic gel and the antimicrobial or other agent, such as silver, is shown in a tank subject to agitation, 200. Next, foam is inserted into the agitating tank, 202. After saturation, the foam is removed and fed through rollers or the like to remove excess solution, 204. The excess solution is captured, 206, and subjected to filtration by a filter sufficiently fine to rid particles from the solution and break apart any chunks of solution that may have formed during the process, 208. A 150-micron filter has been found to be effective during certain silver-solution coating experiments. The filtered solution is then returned to the tank for re-use, 210.

The foam from the removal step 204 is subjected to a convection oven for drying, 212. During certain silver-solution coating experiments, when the temperature of the oven is set at about 90° C., 20 minutes has been found to be an effective drying time. However, it is preferable to dry the foam for about at least 6 minutes to minimize any breakdown of coating. The foam is next packaged in appropriate containers, such as the MVTR pouch or similar containers for shipment to the user, 214.

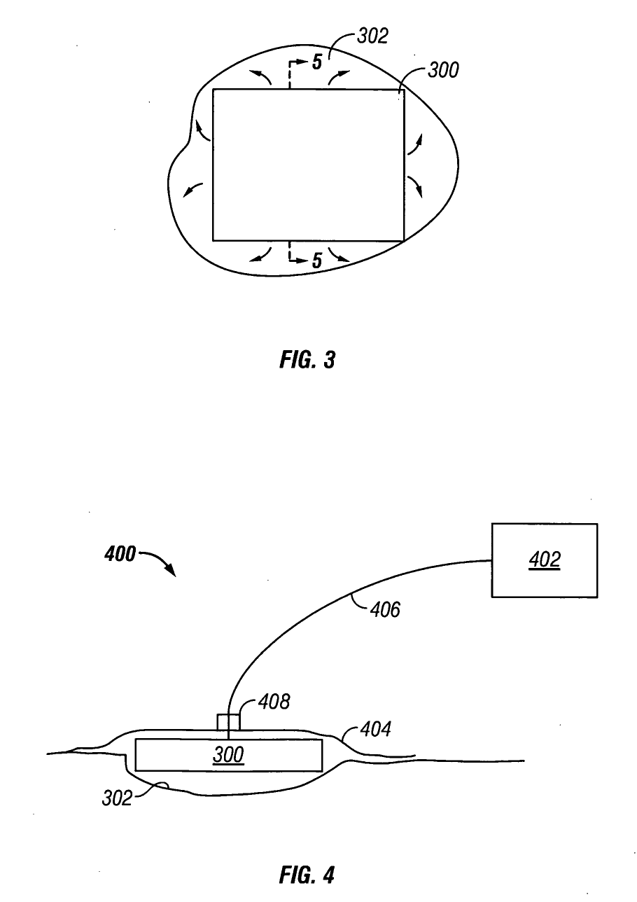

Referring now to FIG. 3, a schematic top plan view of a dressing 300 coated using the process of FIG. 1 as applied to a wound site 302 is shown. As indicated by the arrows, silver ions from the dressing 300 contact the wound site 302 and effectively eliminate bacteria formed thereon.

When used in combination with negative pressure therapeutic devices, such as that made by Kinetic Concepts, Inc., the dressing 300 is particularly effective. FIG. 4 is a side view of the dressing 300 of FIG. 3 on a wound site 302 in combination with a negative pressure therapeutic device 400, which includes a control system 402, a drape 404 for covering the dressing 300 and wound site 302, a vacuum hose 406 connected to the control system 402 and to the wound site 302 through the dressing 300, and a connector 408 for connecting the vacuum hose 406 to the drape 404. Application of negative pressure by the control system 402 through the dressing 300 effectively pulls harmful pathogens through the uniformly coated dressing 300, thereby killing the pathogens. In addition, other surfaces of the dressing 300 in contact with the wound site 302 achieve the same result.

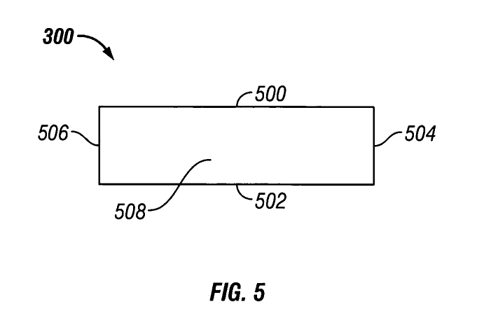

Referring now to FIG. 5, a cross-section of the dressing 300 of FIG. 3 taken along line 5-5 is shown, illustrating the uniform coating of the dressing 300. The dressing 300 has an upper surface 500, a lower surface 502, side surfaces 504, 506 and interior surface 508. All surfaces 500, 502, 504, 506, and 508, are coated with the silver coating, thereby providing an effective barrier to any pathogens that directly contact the surfaces or are indirectly exposed thereto by silver ions migrating away from the dressing 300.

The previous description is of preferred embodiments for implementing the invention, and the scope of the invention should not necessarily be limited by this description. The scope of the present invention is instead defined by the following claims.

We claim:

1. A method for coating a foam for placement on a wound site, the method comprising the steps of:
combining a hydrophilic gel with silver to create a coating solution;
agitating the coating solution in a closed environment;
placing the foam in the closed environment;
saturating the foam with the coating solution;
removing excess solution from the saturated foam; and
drying the saturated foam.

2. The method of claim 1, wherein the foam comprises polyurethane.

3. The method of claim 1, wherein the coating solution comprises a silver hydrogel polymer.

4. The method of claim 3, wherein the coating contains PVP.

5. The method of claim 3, wherein the coating contains Chitosan.

6. The method of claim 3, wherein the coating contains silver sodium aluminosilicate.

7. The method of claim 1, further comprising the step of placing the solution in a holding tank after the step of combining the hydrophilic gel.

8. The method of claim 1, wherein the environment is a dark environment to prevent the foam from changing color.

9. The method of claim 1, wherein the foam comprises reticulated polyurethane die cut.

10. The method of claim 1, wherein the step of saturating the foam with the solution is accomplished through soaking the foam in the solution.

11. The method of claim 1, wherein the step of saturating the foam with the solution is accomplished through squeezing the foam in the solution to allow the foam to absorb the solution.

12. The method of claim 1, further comprising the step of weighing the saturated foam after the step of saturating the foam.

13. The method of claim 12, further comprising the step of weighing the foam a second time after the step of drying the foam.

14. The method of claim 1, wherein the step of drying the saturated foam comprises placing the foam in a convectional forced-air oven set to a predetermined temperature for a predetermined amount of time.

15. The method of claim 1, further comprising packaging the foam in a moisture vapor transmission rate pouch to limit exposure of the foam to light and to humidity.

16. The method of claim 1, further comprising:
placing the foam on a wound site;
covering the wound site with a drape;
connecting a vacuum hose at one end to the foam through the drape and at the other end to a vacuum;
applying negative pressure to the wound site to pull pathogens and other harmful material through the foam to kill the pathogens and harmful material.

17. A method for treating a wound, comprising the steps of:
combining a hydrophilic gel with silver to create a coating solution;
agitating the coating solution in a holding tank;
placing the foam in the holding tank;
saturating the foam with the coating solution by soaking the foam in the coating solution for a predetermined amount of time;
removing excess solution from the foam by rolling the saturated foam through a roller;
drying the foam in a convection oven at a temperature of about 90° C. for at least about 6 minutes to completely dry the foam;
applying the foam to a wound surface;
connecting a vacuum to the foam;
draping the wound surface; and
applying negative pressure to the wound via the vacuum, wherein harmful materials from the wound are neutralized via the coating on the foam.

18. The method of claim 17, wherein the coating solution comprising a debriding agent.

19. The method of claim 17, wherein the coating comprises anesthetizing agents.

20. The method of claim 17, wherein the coating comprises a growth factor.
